(19) United States
(12) Patent Application Publication
(10) Pub. No.: US 2017/0007106 A1
(43) Pub. Date: Jan. 12, 2017

(54) ENDOSCOPE

(71) Applicant: OLYMPUS CORPORATION, Tokyo (JP)

(72) Inventor: Reiji KOYAMA, Tokyo (JP)

(73) Assignee: OLYMPUS CORPORATION, Tokyo (JP)

(21) Appl. No.: 15/269,650

(22) Filed: Sep. 19, 2016

Related U.S. Application Data
(63) Continuation of application No. PCT/JP2015/063898, filed on May 14, 2015.

(30) Foreign Application Priority Data
May 15, 2014 (JP) 2014-101648

(51) Int. Cl. A61B 1/05; A61B 1/12; A61B 1/06; A61B 1/00; A61B 1/005; A61B 1/018

(52) U.S. Cl. CPC A61B 1/05; A61B 1/0051; A61B 1/0052; A61B 1/018; A61B 1/0661; A61B 1/0676; A61B 1/00165; A61B 1/12

(57) ABSTRACT

An endoscope includes a bending section, an operation section including first and second operation sections, a grasping section housing, an exterior housing, a main frame, first and second towing members, first and second towing-member-adjusting sections, a first fixing member for fixing the first-towing-member-adjusting section, and a second fixing member for fixing the second-towing-member-adjusting section. The first and second fixing members are formed by flat plates having a same shape, selected to be fixed to the main frame with one end side directed to the insertion section on the first operation section side or fixed with the other end side directed to the insertion section on the second operation section side, and formed asymmetrically with respect to the center axis orthogonal to a direction in which the first and second towing members extend such that fixed sections are closer to the one end side than the other end side.

### CROSS REFERENCE TO RELATED APPLICATION

This application is a continuation application of PCT/JP2015/063898 filed on May 14, 2015 and claims benefit of Japanese Application No. 2014-101648 filed in Japan on May 15, 2014, the entire contents of which are incorporated herein by this reference.

### BACKGROUND OF THE INVENTION

1. Field of the Invention

The present invention relates to an endoscope including, in an operation section, a bending operation mechanism for bending a bending section.

2. Description of the Related Art

In recent years, insertion devices inserted into subjects/objects, for example, endoscopes are widely used in a medical field and an industrial field. The endoscope used in the medical field is configured such that, by inserting an elongated insertion section into a body cavity, which is a subject, it is possible to observe an organ in the body cavity and perform various kinds of treatment using a treatment instrument inserted into an insert-through channel for the treatment instrument included in the endoscope according to necessity. The endoscope used in the industrial field is configured such that, by inserting an elongated insertion section of the endoscope into an object such as a jet engine or a pipe of a factory, it is possible to perform observation of scratches, corrosion, and the like and inspections of various kinds of treatment and the like of a part to be inspected in the object.

A conventional endoscope is normally configured by an elongated insertion section and an operation section coupled to a proximal end portion of the insertion section. The insertion section is configured by coupling an elongated flexible tube section having flexibility, a bending section, and a distal-end configuring section in order from a proximal end side. On an outer surface of the operation section, besides a bending operation knob, which is an operation member configuring a part of a bending operation mechanism for bending the bending section, various operation members are disposed.

In a usual case, during use of the endoscope, a user or an operator of the endoscope operates the various operation members of the operation section while grasping the operation section using a palm and fingers. Therefore, in the conventional endoscope, operability of the endoscope is taken into account by applying contrivance to the operation section to reduce the operation section in size and form the operation section in an easy-to-grasp shape.

For example, in the operation section of the conventional endoscope, a grasping section grasped by the user is provided. Contrivance for a shape explained below is applied to a shape of the grasping section. That is, in the grasping section of the operation section, a shape is considered taking into account characteristic points of a hand of a human who grasps the grasping section, more specifically, characteristics that, for example, a little finger is short compared with an index finger and a middle finger and a thumb grasps the grasping section from a direction opposite to a direction from which the other four fingers grasp the grasping section. As an example of the shape, for example, in the grasping section of the operation section, a circumferential length of a part grasped by short fingers is set to be shorter than a circumferential length of a part grasped by long fingers. In other words, an axis in a major axis direction extending along a side surface on a side on which the fingers are put is formed to incline at a predetermined angle with respect to an axis in a major axis direction extending along a side surface on a side where the palm is in contact in the grasping section. An inclined line of the axis is set such that a shape of the operation section becomes a tapered shape toward a distal end side.

On the other hand, in constituent members disposed inside the operation section of the endoscope of the normal form, for example, concerning the bending operation mechanism and the like, some constituent member is configured by a pair of constituent members in order to secure bending operation in an up-down direction and bending operation in a left-right direction with respect to the bending section. When such a pair of constituent members is disposed inside the operation section, for example, the pair of constituent members is normally disposed symmetrically with respect to a center axis of the operation section. Shapes of components themselves are often formed to be symmetrical with respect to the center axis. Endoscopes having such a configuration are disclosed by, for example, Japanese Patent Application Laid-Open Publication No. H9-238895 and Japanese Patent Application Laid-Open Publication No. 2009-172019.

On the other hand, when the pair of constituent members is disposed inside the operation section, by using components having completely the same shape as a pair and contriving disposition of the components, it is possible to contribute to a reduction in the number of component types and a reduction in manufacturing cost.

### SUMMARY OF THE INVENTION

An endoscope in an aspect of the present invention includes: a bendable bending section provided in an insertion section inserted into a subject; an operation section connected to a proximal end portion of the insertion section and grasped by an operator, a region of the operation section being divided into a first operation section side and a second operation section side across a center axis extending in a longitudinal direction; a grasping section housing configuring a portion adjacent to the insertion section side in the operation section and grasped by the operator; an exterior housing configuring a portion located on an opposite side of the insertion section side across the grasping section housing in the operation section, a dimension of the exterior housing along the center axis on the first operation section side being set longer than a dimension of the exterior housing along the center axis on the second operation section side, an internal constituent unit for bending the bending section being disposed on an inside of the exterior housing; a main frame provided across the center axis in the operation section to fix the internal constituent unit; a first towing member and a second towing member respectively disposed on the first operation section side and the second operation section side and made movable along the center axis with respect to the main frame in order to bend the bending section; a first-towing-member adjusting section and a second-towing-member adjusting section respectively disposed on the first operation section side and the second operation section side and configured to adjust moving ranges along the center axis respectively in the first towing member and the second towing member; a first fixing member fixed to the main frame on the first operation section side to fix the first-towing-member adjusting section to the main frame on the first operation section side; and a second fixing member fixed to the main frame on the second operation section side to fix the second-towing-member adjusting section to the main frame on the second operation section side. The first fixing member and the second fixing member are formed by flat plates having a same shape extending along respective moving directions of the first towing member and the second towing member, selected to be fixed to the main frame with one end side directed to the insertion section on the first operation section side or fixed to the main frame with another end side directed to the insertion section on the second operation section side, and formed asymmetrically with respect to a center axis orthogonal to a direction in which the first towing member and the second towing member extend such that fixed sections fixed to the main frame are closer to the one end side than the other end side.

### BRIEF DESCRIPTION OF THE DRAWINGS

FIG. 1 is an overall configuration diagram showing one side surface (a side surface on which an operation knob is disposed) of an endoscope according to an embodiment of the present invention;

FIG. 2 is an overall configuration diagram showing the other side surface (a side surface disposed to be opposed to the side surface on the operation knob side) of the endoscope according to the embodiment of the present invention;

FIG. 3 is a main part enlarged sectional view showing a part of an internal configuration of an operation section in the embodiment shown in FIG. 1;

FIG. 4 is an arrow view viewed from an arrow [4] direction in FIG. 3;

FIG. 5 is an arrow view viewed from an arrow [5] direction of FIG. 3;

FIG. 6 is a main part enlarged perspective view extracting a main frame of the operation section of the endoscope shown in FIG. 1 and showing a part of the main frame;

FIG. 7 is a main part enlarged perspective view showing a state in which a part of constituent members of a bending operation mechanism is attached to the main frame shown in FIG. 6;

FIG. 8 is a main part enlarged sectional view showing a part of the bending operation mechanism of the operation section of the endoscope shown in FIG. 1;

FIG. 9 is a main part enlarged exploded perspective view showing main constituent members of the bending-angle adjusting mechanism in the endoscope shown in FIG. 1;

FIG. 10 is a three-plane drawing conceptually showing a shape of a guide block in the bending-angle adjusting mechanism shown in FIG. 9;

FIG. 11 is a four-plane drawing conceptually showing a shape of a bearing plate in the bending-angle adjusting mechanism shown in FIG. 9;

FIG. 12 is a diagram conceptually showing disposition of the bending operation mechanism and the bending-angle adjusting mechanism disposed on an inside of the operation section in the endoscope shown in FIG. 1;

FIG. 13 is a main part enlarged assembled perspective view viewed from an arrow [13] direction of a state in which the bending-angle adjusting mechanism shown in FIG. 9 is assembled;

FIG. 14 is a main part enlarged assembled perspective view viewed from an arrow [14] direction of the state in which the bending-angle adjusting mechanism shown in FIG. 9 is assembled;

FIG. 15 is a main part enlarged view enlarging and showing a part (mainly a coupling structure of a chain and a coupling member) of the bending-angle adjusting mechanism shown in FIG. 9;

FIG. 16 is a plan view showing one side surface in the bending-angle adjusting mechanism shown in FIG. 9;

FIG. 17 is a sectional view taken along line [17]-[17] shown in FIG. 16;

FIG. 18 is a main part enlarged perspective view enlarging and showing a part of the guide block among the constituent members of the bending-angle adjusting mechanism shown in FIG. 9; and

FIG. 19 is a main part enlarged perspective view showing a state in which two adjustment shafts are built into the bearing plate which is assembled to the guide block shown in FIG. 18 and fixed by a fixing pin.

### DETAILED DESCRIPTION OF THE PREFERRED EMBODIMENT(S)

The present invention is explained below with reference to an embodiment shown in the figures. In the respective drawings used for the following explanation, scales are sometimes differentiated for each of respective constituent elements and shown in order to show the respective constituent elements in recognizable seizes on the drawings. Therefore, in the present invention, the numbers of constituent elements, shapes of the constituent elements, ratios of sizes of the constituent elements, and relative positional relations of the respective constituent elements described in the drawings are not limited only to forms shown in the figures.

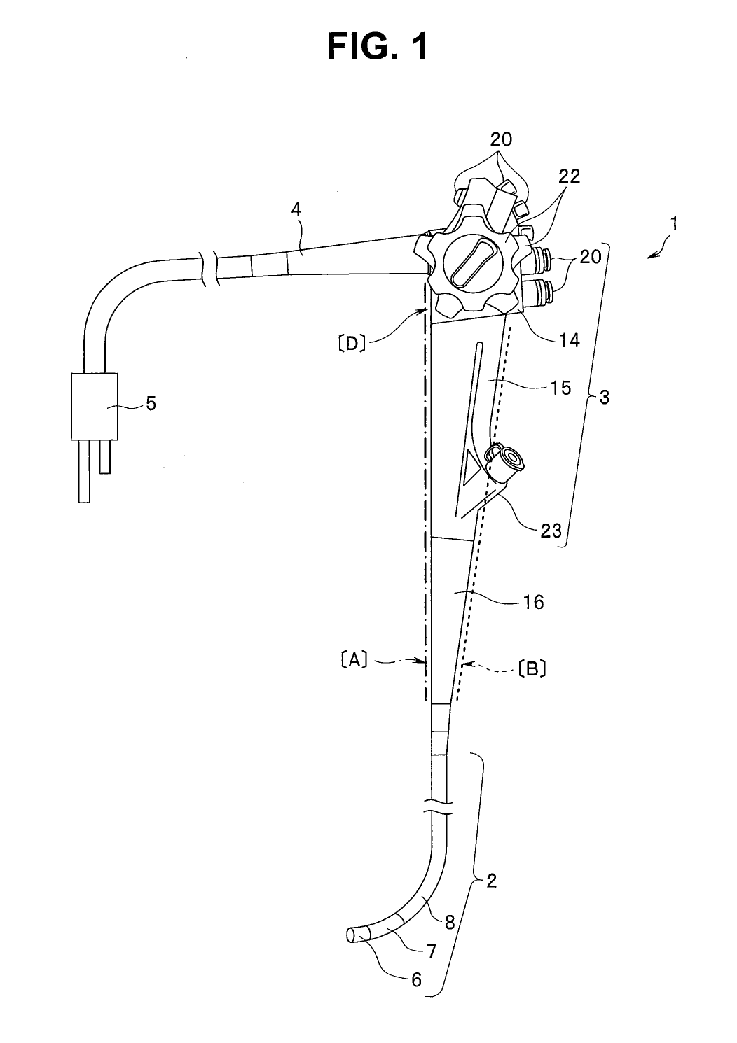

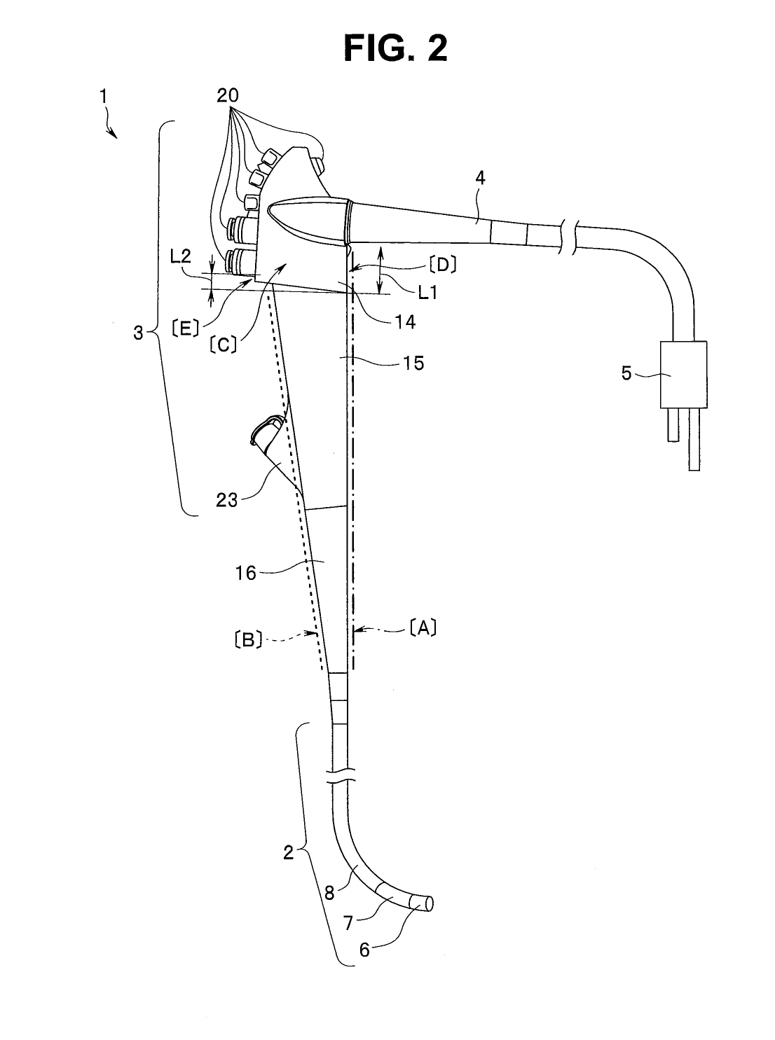

First, an overview of an overall configuration of an endoscope in an embodiment of the present invention is explained below with reference to FIG. 1 and FIG. 2. FIG. 1 and FIG. 2 are exterior views showing an overall configuration of the endoscope in the embodiment of the present invention. FIG. 1 shows one side surface (a side surface on which an operation knob is disposed) in the endoscope. FIG. 2 shows the other side surface (a side surface disposed to be opposed to the side surface on the operation knob side) of the endoscope.

An endoscope 1 in the present embodiment is mainly configured by, as shown in FIG. 1 and FIG. 2, an elongated-shaped insertion section 2 inserted into a body cavity (a subject), an operation section 3 concatenated to a proximal end side of the insertion section 2, a universal cord 4 extended to an outside with a proximal end portion coupled to one side surface of the operation section 3, a connector 5 disposed at a distal end portion of the universal cord 4, and the like.

The insertion section 2 in the endoscope 1 is configured by coupling a rigid distal-end configuring section 6, a bending section 7, and an elongated-shaped flexible tube section 8 having flexibility in order from a distal end. The bending section 7 is configured to be bendable in each of four directions of upward and down ward directions and left and right directions. Bending operations of the bending section 7 in any directions are enabled by combining bending operations in the four directions. Note that, as an internal constituent unit for bending the bending section 7, a bending operation mechanism 30 is disposed on an inside of the operation section 3. A detailed configuration of the bending operation mechanism 30 is explained below.

An objective lens, an illumination lens, a washing nozzle, a treatment instrument channel opening, and the like are disposed on a distal end face of the distal-end configuring section 6. On an inside of the distal-end configuring section 6, besides an image pickup device, electric components and the like such as an electric board, a video cable extended from the image pickup device, and the like, air feeding conduit, a water feeding conduit, and the like coupled to the washing nozzle, a light guide fiber for supplying illumination light to the illumination lens, and the like are disposed (not shown in the figure). The video cable and the light guide fiber are inserted through the insertion section 2, the operation section 3, and the universal cord 4 and concatenated to the connector 5. The air feeding conduit and the water feeding conduit are inserted through the insertion section 2 and concatenated to the connector 5 through an air/water feeding cylinder provided in the operation section 3 and the universal cord 4.

Note that, concerning outer surface and internal configurations of the distal-end configuring section 6, assuming that outer surface and internal configurations same as outer surface and internal configurations of the conventional endoscope of the general form are provided, detailed explanation and illustration of the external surface and internal configurations are omitted.

The operation section 3 is a housing member in which an exterior housing 14 and a grasping section housing 15, which are parts grasped by a user during use of the endoscope 1, are integrally and water-tightly formed. A proximal end portion of the insertion section 2 is concatenated from one end portion of the grasping section housing 15. In this case, in a joint portion of the one end portion of the grasping section housing 15 and the proximal end portion of the insertion section 2, a bend preventing portion 16 formed of an elastic rubber member or the like for suppressing sudden bending of the flexible tube section 8 of the insertion section 2 is provided. That is, the grasping section housing 15 is concatenated to the exterior housing 14 in a longitudinal direction of the operation section 3.

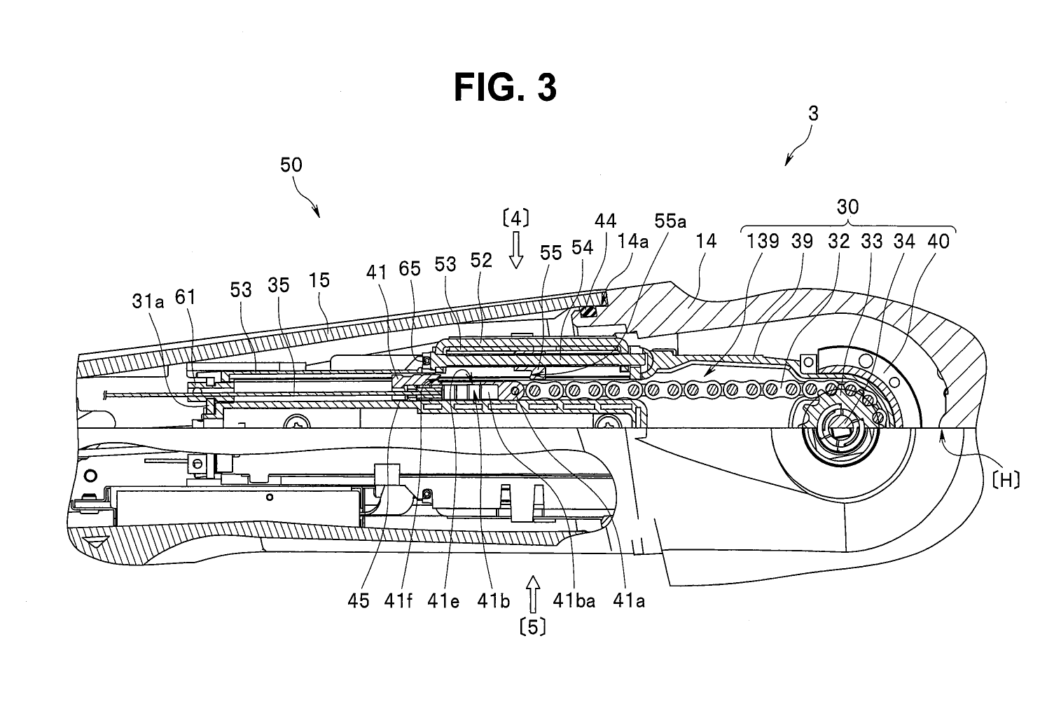

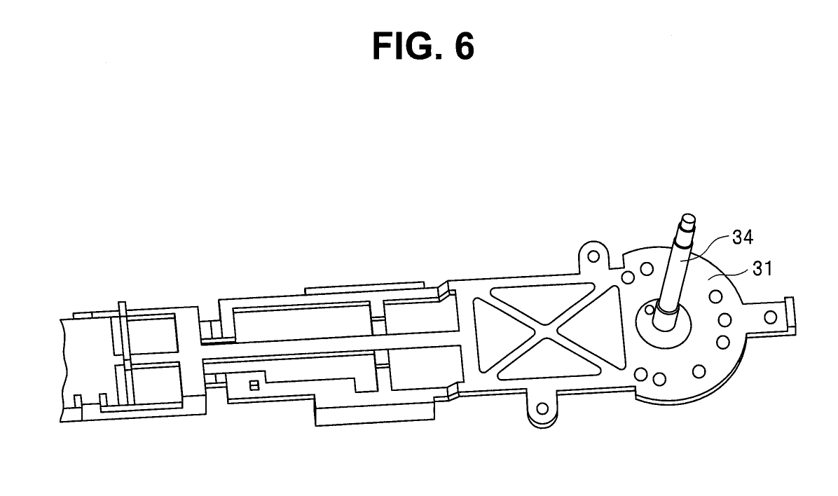

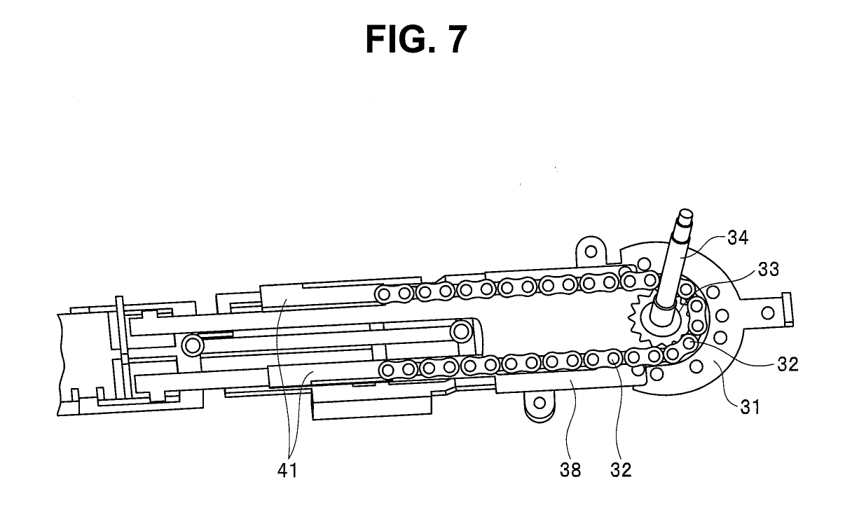

In the exterior housing 14 of the operation section 3, a plurality of bending operation knobs 22, which are operation members for bending the bending section 7 of the insertion section 2, are rotatably disposed coaxially with a support shaft 34 (not shown in FIG. 1; see FIG. 3, FIG. 6, and FIG. 7), which is a shaft member (a detailed configuration is explained below). The plurality of bending operation knobs 22 are mechanically connected to a bending operation mechanism 30 (not shown in FIG. 1; details are explained below; see FIG. 3 and the like) disposed on the inside of the operation section 3, that is, insides of the exterior housing 14 and the grasping section housing 15.

On an outer surface of the exterior housing 14 of the operation section 3, various operation members, for example, a plurality of operation members 20 for remotely operating peripheral apparatuses such as a video processor are provided. Further, on an outer surface of the grasping section housing 15, a treatment-instrument lead-in port 23 for leading in a not-shown treatment instrument and the like is provided. The treatment-instrument lead-in port 23 communicates with a treatment instrument channel on an inside. The treatment instrument channel is inserted through an inside of the insertion section 2 to the treatment instrument channel opening of the distal-end configuring section 6.

Note that the endoscope 1 is connected to a not-shown light source device and a control device such as a video processor via the connector 5 and configured to operate as an endoscope system.

When the endoscope 1 in the present embodiment configured as explained above is used, the user operates various operation members provided in the operation section 3 while grasping a predetermined part of the operation section 3 using, for example, a palm and fingers of a left hand.

More specifically, for example, if a palm portion of the user is put near a part indicated by a sign [D] in FIG. 1 and FIG. 2, pad side parts (near second and third joints) in middles of fingers other than a thumb among the fingers are put near a part indicated by a sign [C] in FIG. 2 and a pad side part of the thumb is put near a part indicated by a sign 14 in FIG. 1. At this point, tip portions of the respective fingers other than the thumb are present in positions where the fingers can operate the plurality of operation members 20 and the thumb is present in a position where the thumb can operate the bending operation knobs 22. In this way, during the use of the endoscope 1, the user grasps a part of the operation section 3, that is, outer surface sides of the exterior housing 14 and the grasping section housing 15 using the fingers of the left hand and performs various kinds of operation.

Therefore, as shown in FIG. 1 and FIG. 2, a shape of the operation section 3 in the endoscope 1 in the present embodiment is formed such that, when the user grasps the operation section 3, an axis (see a dotted line indicated by a sign [B]) in a major axis direction extending along a side surface on a side on which the figures are put inclines at a predetermined angle with respect to an axis (see an alternate long and short dash line indicated by a sign [A]) in a major axis direction extending along a side surface on a side on which a part where the palm is put is present. The operation section 3 in that case is set to be a tapered shape toward a distal end side of the operation section 3. By forming the operation section 3 in such a shape, in a grasped portion of the operation section 3, a circumferential length of a part on a side grasped by short fingers is set to be shorter than a circumferential length of a part on a side grasped by long fingers.

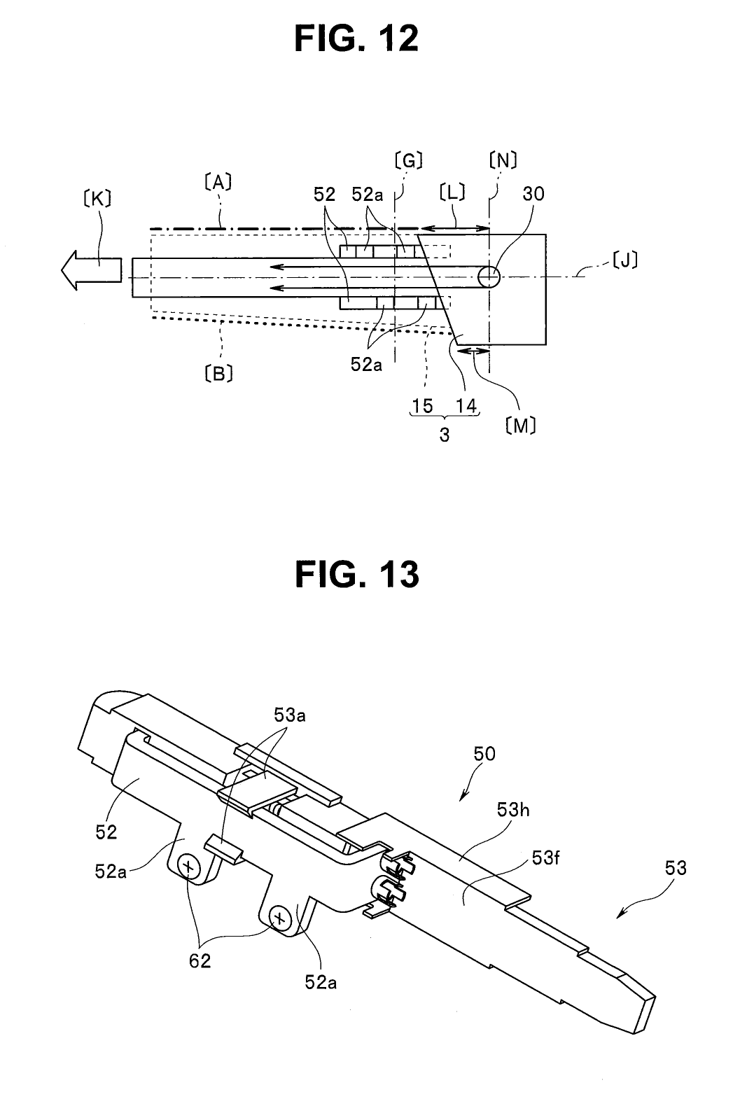

Note that a part including the axis [A] shown in FIG. 1 and FIG. 2 in the operation section 3 is referred to as first grasping section. The first grasping section is a part on one side surface of the grasping section housing 15 formed to cover a part of a main frame 31 and is a part for the user and the operator to grasp. Similarly, a part including the axis [B] shown in FIG. 1 and FIG. 2 in the operation section 3 is referred to as second grasping section. The second grasping section is a part on the other side surface of the grasping section housing 15 formed to cover a part of the main frame 31 and is a part opposed to the first grasping section. The second grasping section is formed shorter than the first grasping section in a longitudinal axis direction. Note that, in this relation, a part on the outer surface of the exterior housing 14 of the operation section 3 extending along a line [L] shown in FIG. 12 explained below as a line on an extended line of the line [A] is referred to as first operation section. The first operation section is a part on one side surface of the exterior housing 14 formed to cover a part of the main frame 31 and is a part for the user and the operator to operate. That is, the first operation section forms one side of the operation section 3 with respect to a center axis (explained below) of the operation section 3. Similarly, a part on the outer surface of the exterior housing 14 of the operation section 3 on the axis [B] side, that is, a part opposed to the first operation section (a part indicated by a line [M] shown in FIG. 12) is referred to as second operation section. That is, the second operation section forms the other side of the operation section 3 with respect to the center axis (explained below) of the operation section 3. The second operation section is formed shorter than the first operation section in the longitudinal axis direction of the operation section 3.

As shown in FIG. 2, a dimension L2 in an axial direction of a region (a finger tip contact part) indicated by a sign [E] is set to be smaller than a dimension L1 in an axial direction of a region (a palm contact part) indicated by a sign [D]. A region (a finger pad contact part) indicated by a sign [C] is set to be able to be secured as wide as possible.

A boundary line in a connecting portion between the exterior housing 14 and the grasping section housing 15 in the operation section 3 is formed to have inclination with respect to the axis [A] as shown in FIG. 1, FIG. 2, and the like, for example, when viewed from a side surface.

In the endoscope 1 in the present embodiment configured as explained above, various constituent members, constituent units, and the like are housed and disposed on the inside of the operation section 3. Among the constituent members and the constituent units, as a main constituent unit, there is the bending operation mechanism 30 for bending the bending section 7 in association with rotational operation of the bending operation knobs 22 by the user. A configuration of the bending operation mechanism 30 is explained below with reference to the drawings.

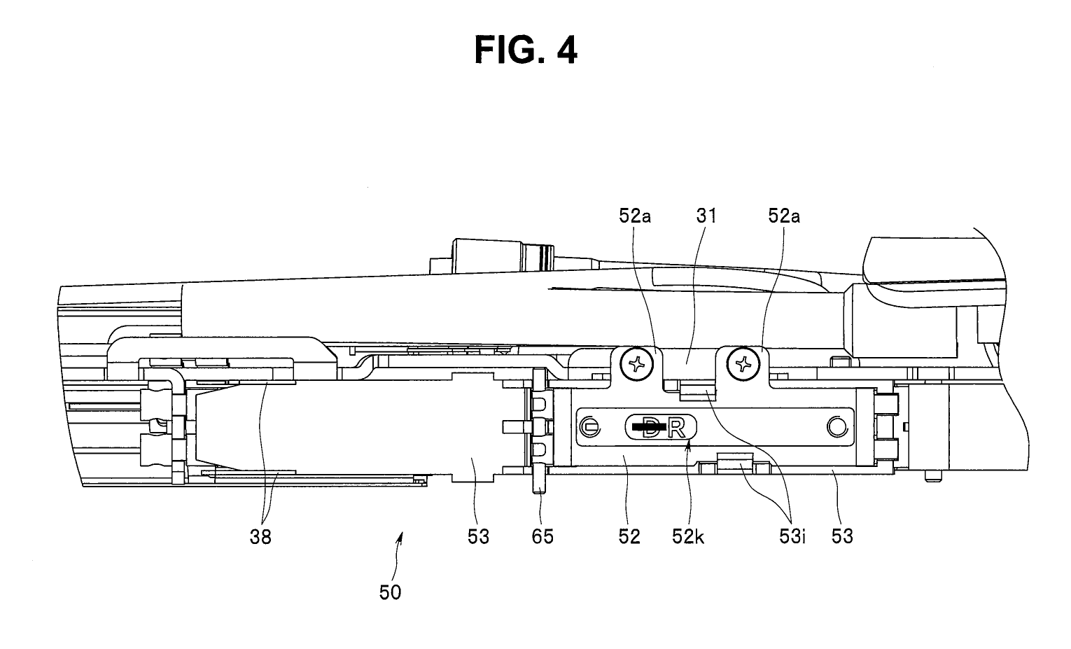

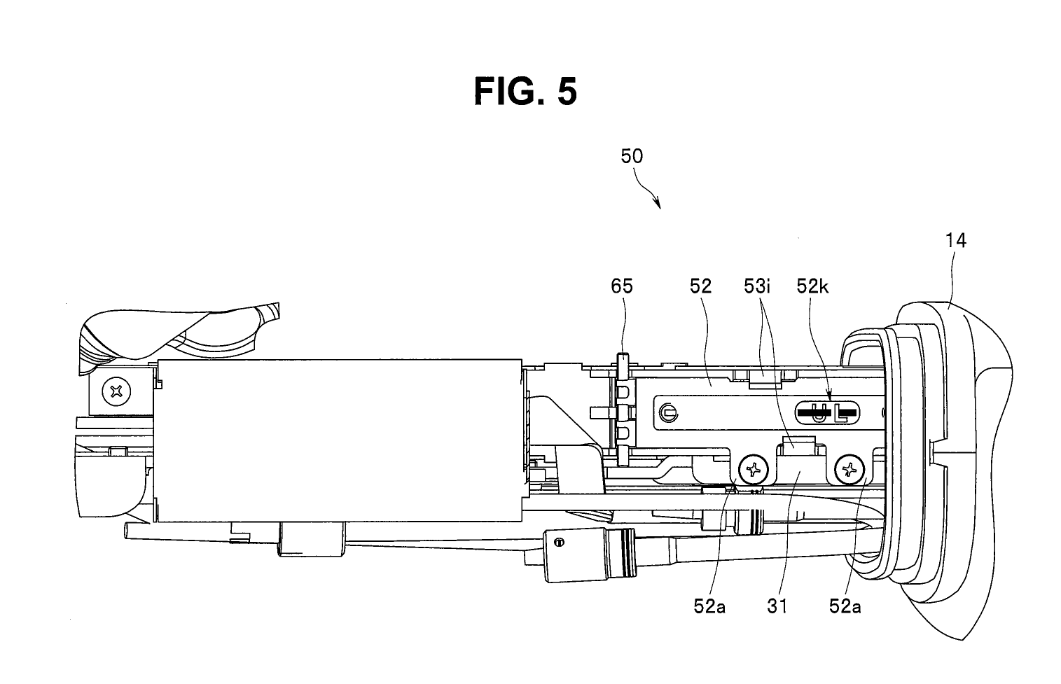

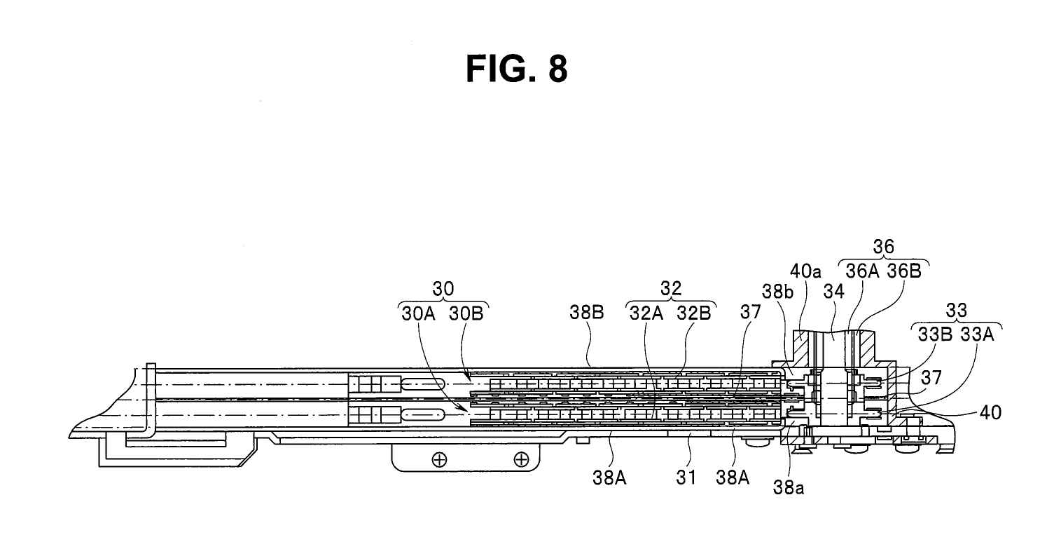

FIG. 3 is main part enlarged sectional view showing a part of an internal configuration of the operation section in the endoscope shown in FIG. 1. FIG. 4 is an arrow view viewed from an arrow [4] direction in FIG. 3. FIG. 5 is an arrow view viewed from an arrow [5] direction in FIG. 3. Note that, in FIG. 4 and FIG. 5, illustration of exterior components is omitted to show the internal configuration of the operation section 3. FIG. 6 is a main part enlarged perspective view extracting the main frame of the operation section of the endoscope shown in FIG. 1 and showing a part of the main frame. FIG. 7 is a main part enlarged perspective view showing a state in which a part of constituent members of the bending operation mechanism is attached to the main frame shown in FIG. 6. FIG. 8 is a main part enlarged sectional view showing a part of the bending operation mechanism of the operation section of the endoscope shown in FIG. 1.

On the inside of the operation section 3, the bending operation mechanism 30 is disposed as shown in FIG. 3. The bending operation mechanism 30 is configured to be capable of rotating a sprocket 33, which is a rotary ring attached to the support shaft 34, by rotating the bending operation knobs 22 attached to the support shaft 34, which is a rotating shaft provided in the operation section 3. A chain 32, which is a long member, is wound on the sprocket 33. A bending operation wire 35 extended from the bending section 7 of the insertion section 2 is connected to both ends of the chain 32. With this configuration, when the bending operation knobs 22 are rotated in forward and reverse directions, the chain 32 and the bending operation wire 35 reciprocatingly move in the major axis direction of the operation section 3. The bending section 7 is configured to perform a bending motion in response to the reciprocating movement.

The respective constituent members of the bending operation mechanism 30 are fixed to the main frame 31 (not shown in FIG. 3; see FIG. 4 to FIG. 6 and the like) disposed on the inside of the operation section 3. The main frame 31 is, for example, as shown in FIG. 6, a ladder structure formed to have a longitudinal axis in the major axis direction or, although not shown in the figure, a structure configured by a plate structure and is formed by a metal member or the like by, for example, die cast for injection molding aluminum. The main frame 31 is screwed and fixed on the insides of the exterior housing 14 and the grasping section housing 15.

The bending operation mechanism 30 is configured by a left-right bending operation mechanism 30A capable of bending the bending section 7 in a left-right direction, an up-down bending operation mechanism 30B capable of bending the bending section 7 in an up-down direction, and the like (see FIG. 8). As shown in FIG. 8, on an upper surface of the main frame 31, the left-right bending operation mechanism 30A is disposed across a lower chain cover 38A. The up-down bending operation mechanism 30B is disposed across a partition plate 37 on an upper side of the left-right bending operation mechanism 30A. An upper chain cover 38B is disposed on an upper surface of the up-down bending operation mechanism 30B. Note that the left-right bending operation mechanism 30A and the up-down bending operation mechanism 30B have substantially the same configurations.

Respective constituent units of the bending operation mechanisms 30 (30A, 30B) are configured to include, for example, besides constituent members such as the chains 32 (32A, 32B), the sprockets 33 (33A, 33B), the support shaft 34, cylindrical bodies 36 (36A, 36B), the bending operation wire 35 (not shown in FIG. 8), the partition plate 37, and the chain covers 38 (38A, 38B), a bending-angle adjusting mechanism 50 (a detailed configuration is explained below), which is a towing-member adjusting section, and the like. That is, the towing-member adjusting section is disposed on the inside of the operation section 3.

A lower end of the support shaft 34 is planted in the main frame 31 (see FIG. 7 and FIG. 8). On the other hand, an upper end of the support shaft 34 projects to an outside of the exterior housing 14 piercing through the upper chain cover 38B.

The chain covers 38 (38A, 38B) are formed using, for example, thin plate-like members made of metal or resin. As shown in FIG. 8, in positions corresponding to traveling portions of the chains 32 (32A, 32B), two chain covers 38 are respectively disposed in predetermined parts to respectively cover upper and lower surfaces of the two chains 32 (32A, 32B). Note that, as a form of the chain covers 38 (38A, 38B), a reduction in weight is taken into account by using the thin plate-like members.

A left-right cylindrical body 36A is rotatably disposed on an outer circumferential surface of the support shaft 34. An up-down cylindrical body 36B is rotatably disposed on an outer circumferential surface of the left-right cylindrical body 36A. In this case, the left-right cylindrical body 36A and the up-down cylindrical body 36B are capable of rotating independently from each other. A left-right bending operation knob 22 (not shown in FIG. 8; see FIG. 1) is fixed at an upper end of the left-right cylindrical body 36A. An up-down bending operation knob 22 (no shown in FIG. 8; see FIG. 1) is fixed at an upper end of the up-down cylindrical body 36B. Further, two sprockets 33 are rotatably disposed in parts close to a lower end of the support shaft 34, that is, a left-right sprocket 33A of the left-right bending operation mechanism 30A is rotatably disposed on a lower side and an up-down sprocket 33B of the up-down bending operation mechanism 30B is rotatably disposed on an upper side. In this case, the left-right sprocket 33A is fixed at a lower end of the left-right cylindrical body 36A on an inner side. The up-down sprocket 33B is fixed at a lower end of the up-down cylindrical body 36B on an outer side (see FIG. 8; in FIG. 7, only one is shown as a reference sign 33).

That is, a plurality of sprockets 33 (rotary rings) are provided along the longitudinal direction of the support shaft 34 (the rotating shaft). The respective chains 32 (32A, 32B) connected to the bending operation wire 35 are respectively wound on the respective sprockets 33 (33A, 33B; the rotary rings). With such a configuration, the left-right sprocket 33A is coupled to one left-right bending operation knob 22 via the left-right cylindrical body 36A. The up-down sprocket 33B is coupled to the other up-down bending operation knob 22 via the up-down cylindrical body 36B.

The left-right chain 32A meshes with the left-right sprocket 33A. The up-down chain 32B meshes with the up-down sprocket 33B. In the bending operation mechanism 30 of the endoscope 1 in the present embodiment, in order to suppress the respective sprockets 33A and 33B from moving in the axial direction of the support shaft 34 and restricting positions of the sprockets 33A and 33B in predetermined positions on the support shaft 34 and suppress the respective chains 32A and 32B from being wound into the respective sprockets 33A and 33B, holding sections (38a, 38b; see FIG. 8) are formed.

The holding sections (38a, 38b) are parts integrally formed in a form in which predetermined parts of the chain covers 38 are bent. That is, a first holding section 38a is formed in a predetermined part of the lower chain cover 38A opposed to a lower surface side of the left-right sprocket 33A, that is, a part close to the insertion section 2 with respect to the support shaft 34. A second holding section 38b is formed in a predetermined part of the upper chain cover 38B opposed to an upper surface side of the up-down sprocket 33B, that is, a part close to the insertion section 2 with respect to the support shaft 34.

Further, as shown in FIG. 3, in order to suppress the two chains 32 from projecting to sides and form a space section 139 (see FIG. 3) for absorbing slack of the two chains 32 that occurs during bending operation, a sidewall part 39 is formed in a side edge section of the partition plate 37 (see FIG. 3). The sidewall part 39 is formed integrally with the partition plate 37 by, for example, resin outsert molding.

As shown in FIG. 3 and FIG. 8, on outer circumferential sections of the two sprockets 33, cover members 40 for suppressing the two chains 32 from coming off are screwed and fixed to the main frame 31. Cylinder sections 40a (not shown in FIG. 3; shown in only FIG. 8), into which the two cylindrical bodies 36 are inserted, are screwed and fixed to the cover members 40.

Coupling members 41 are fixed at end portions of the respective chains 32 (see FIG. 3 and FIG. 6). A proximal end portion of the bending operation wire 35 inserted through the insertion section 2 is concatenated to the coupling members 41. A distal end portion of the bending operation wire 35 is fixed to a distal end part (not shown in the figure) of the bending section 7 of the insertion section 2 as explained above. Note that the bending operation wire 35 is manufactured by twining a plurality of wires. A stranded wire having flexibility is used as the bending operation wire 35.

The bending operation wire 35 extends to the distal end side of the insertion section 2 after being inserted through a coil pipe 61 (see FIG. 3) near a distal end portion of a guide block 53 of the bending-angle adjusting mechanism 50 (see FIG. 3). The coil pipe 61 is configured from a tubular member having an inner diameter set slightly larger than a diameter of the bending operation wire 35. The coil pipe 61 is fit and fixed to a coil-pipe fixing section 31a (see FIG. 3) provided in the main frame 31. The coil-pipe fixing section 31a is a part formed integrally with the main frame 31. The coil-pipe fixing section 31a is a part configured to be capable of elastically grasping an outer diameter portion of the coil pipe 61. The coil pipe 61 is a constituent member that plays a role of suppressing disarrangement of the bending operation wire 35 slacked by bending operation.

With such a configuration, when the left-right bending operation knob 22 is rotated, the rotation is transmitted to the left-right sprocket 33A via the left-right cylindrical body 36A. The left-right sprocket 33A rotates in the same direction together with the left-right bending operation knob 22. When the left-right sprocket 33A rotates, the left-right chain 32A is driven to travel according to the rotation. The bending operation wire 35 coupled to the left-right chain 32A via the coupling member 41 is driven to be towed according to the traveling of the left-right chain 32A. Therefore, the bending section 7 is bent in the left or right direction. That is, the bending operation wire 35 is a towing member that moves in order to bend the bending section 7.

Similarly, when the up-down bending operation knob 22 is rotated, the rotation is transmitted to the up-down sprocket 33B via the up-down cylindrical body 36B. The up-down sprocket 33B rotates together with the up-down bending operation knob 22. When the up-down sprocket 33B rotates, the up-down chain 32B is driven to travel according to the rotation. The bending operation wire 35 coupled to the up-down chain 32B via the coupling member 41 is driven to be towed according to the traveling of the up-down chain 32B. Therefore, the bending section 7 is bent in the upward or downward direction. That is, the bending operation wire 35 is a towing member that moves in order to bend the bending section 7.

Note that, as shown in FIG. 3, a ring-like fitting concave section 14a, which fits with the proximal end side of the grasping section housing 15, is formed in a distal end part of the exterior housing 14. The proximal end side of the grasping section housing 15 is fit in the fitting concave section 14a of the exterior housing 14. The grasping section housing 15 and the exterior housing 14 are coupled in a state in which an end face and an inner surface of the grasping section housing 15 are in contact with the fitting concave section 14a of the exterior housing 14. An O-shaped ring 44 is attached to a contact section between the proximal end side of the grasping section housing 15 and the fitting concave section 14a of the exterior housing 14. The O-shaped ring 44 is means for water-tightly sealing a contact part between the exterior housing 14 and the grasping section housing 15.

On the other hand, a coupling section between the coupling member 41 and the bending operation wire 35 is configured as explained below. That is, on the proximal end side of the coupling member 41, as shown in FIG. 3, a coupling section 41a (not shown in FIG. 15) coupled to ends of the chains 32 is formed.

In the coupling member 41, a wire locking section 41b that unlockably locks the proximal end portion of the bending operation wire 35 is provided. In the wire locking section 41b, an engaging space 41ba including an opening on one side surface of the coupling member 41 is formed. In the engaging space 41ba, a plurality of engaging convex sections 41c (not shown in FIG. 3; see FIG. 15 for details) projected toward an inner side are provided in parallel along the axial direction of the bending operation wire 35. As shown in FIG. 15, in the engaging space 41ba, by concatenating a plurality of circular grooves 41d along the axial direction of the bending operation wire 35, the engaging convex sections 41c are formed by concatenated portions among the circular grooves 41d adjacent to one another.

On the other side surface (a bottom surface side of the engaging space 41ba) of the coupling member 41, a long hole 41e (see FIG. 15) extended along the axial direction of the bending operation wire 35 and a convex section 41f formed in a substantially claw shape projecting in a direction orthogonal to the axial direction of the coupling member 41 and outward and projecting toward the proximal end side are formed. The long hole 41e is formed in a part close to the coupling section 41a to the chains 32 and is set to have width smaller than width of the engaging space 41ba. The convex section 41f is formed further on a coupling section side to the bending operation wire 35 than the long hole 41e. The convex section 41f comes into contact and engages with concave sections 55a of stoppers 55 explained below, whereby movement of the coupling member 41 in the axial direction of the bending operation wire 35 is restricted.

A locking member 45 formed in a drum shape is fixed at the proximal end portion of the bending operation wire 35 using fixing means such as soldering, brazing, or caulking. When the locking member 45 is inserted into any one of the plurality of circular grooves 41d, the locking member 45 engages with corresponding one of the plurality of engaging convex sections 41c. In this state, the locking member 45 is engaged and fixed in the engaging space 41ba. In this way, the bending operation wire 35 is coupled to the chains 32 via the coupling members 41. In this case, according to into which of the plurality of circular grooves 41d the locking member 45 is inserted, it is possible to adjust a fixing position of the bending operation wire 35 in the axial direction. Note that, as shown in FIG. 3, the movement in the axial direction of the coupling members 41 is guided by the guide block 53 of the bending-angle adjusting mechanism 50 and a guide wall (not shown in the figure) formed in the main frame 31.

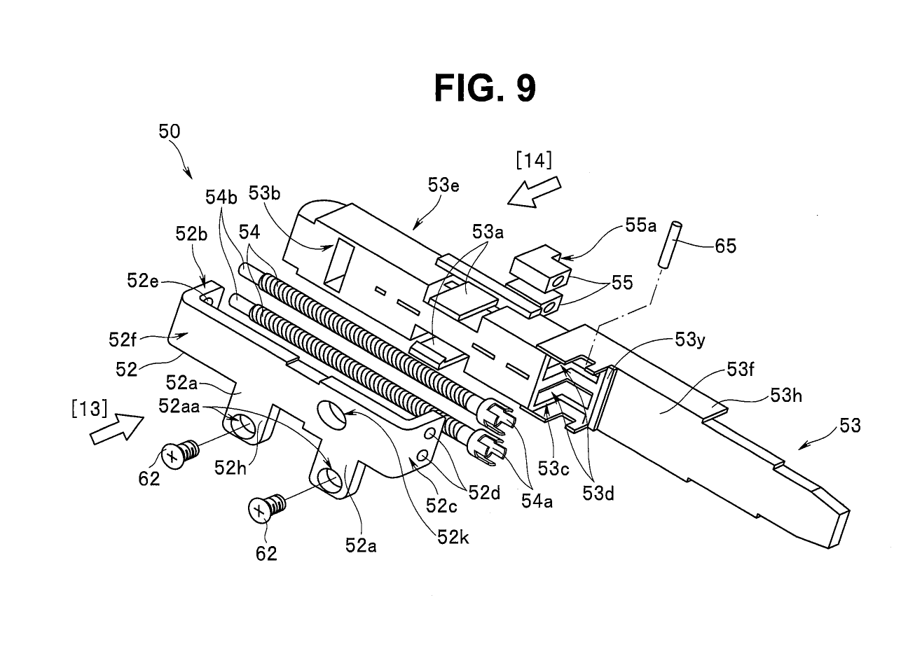

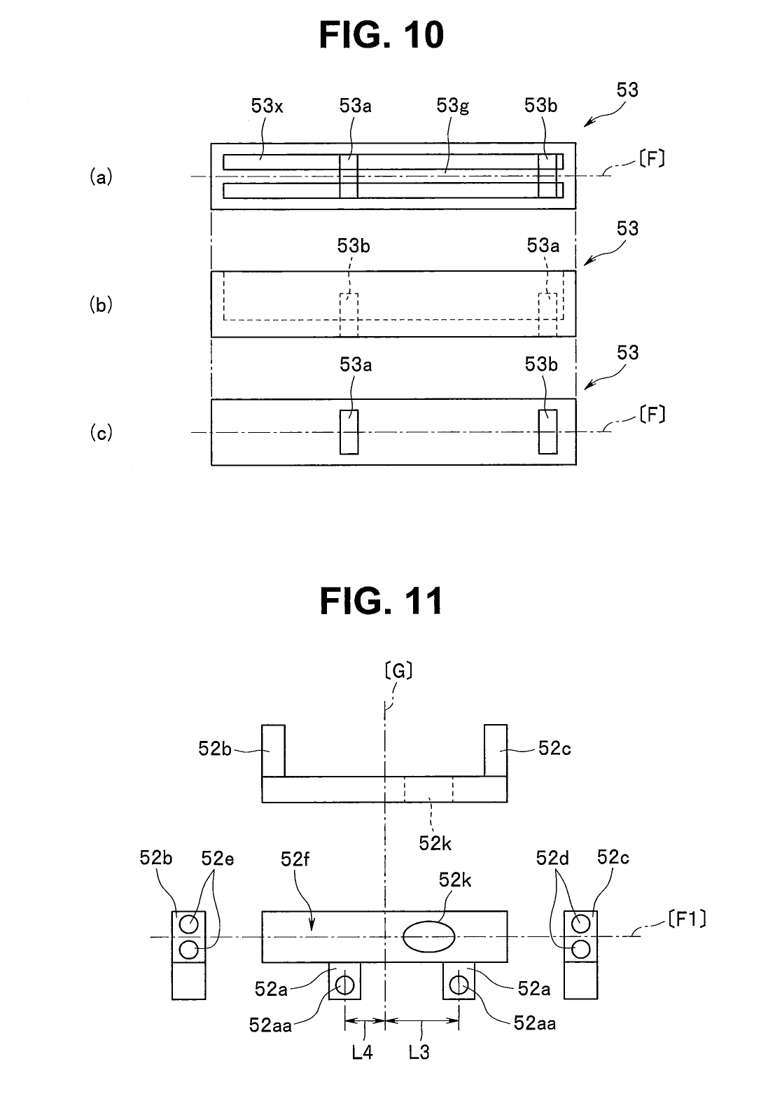

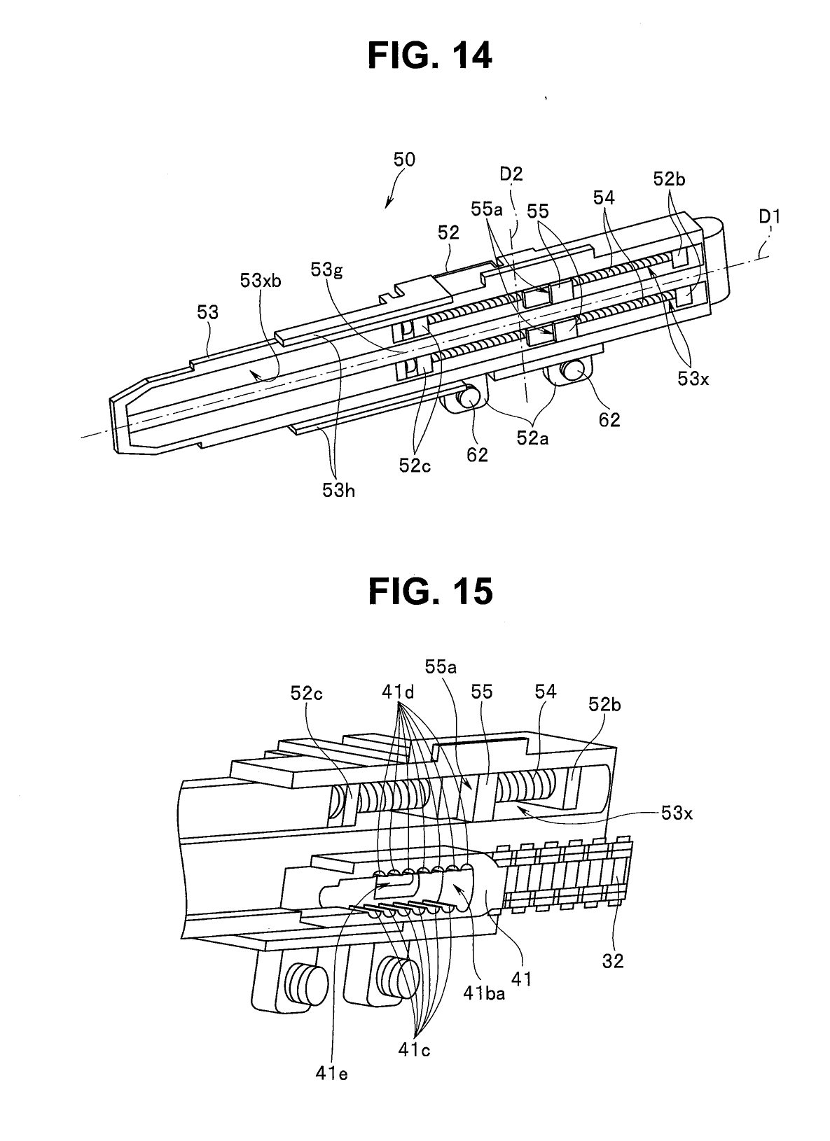

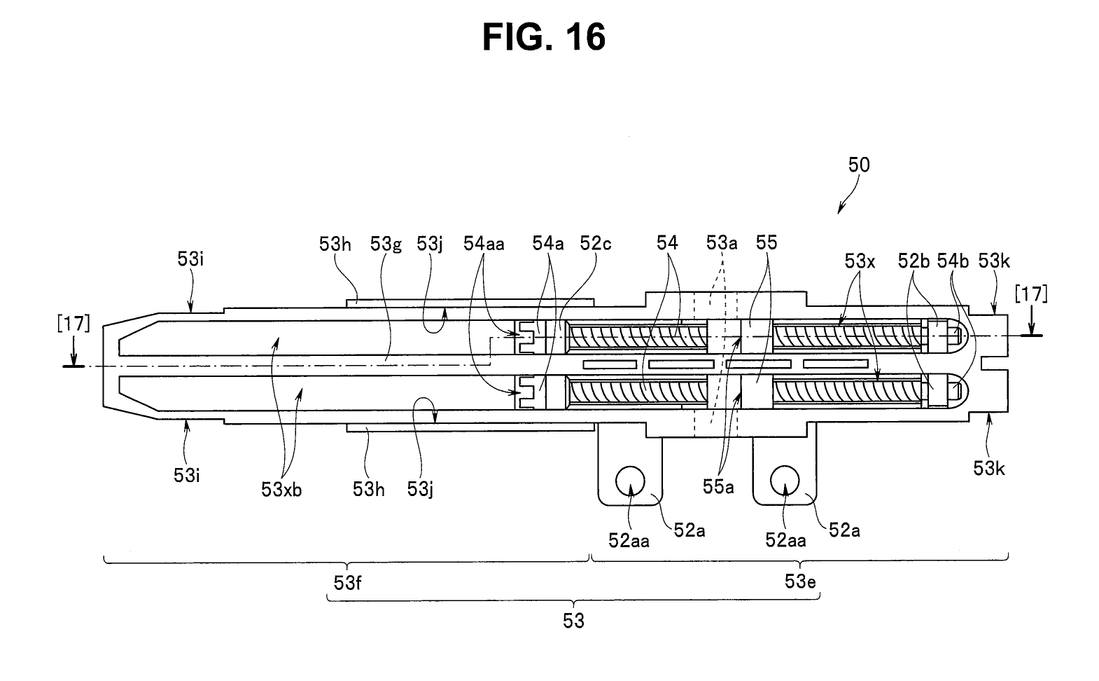

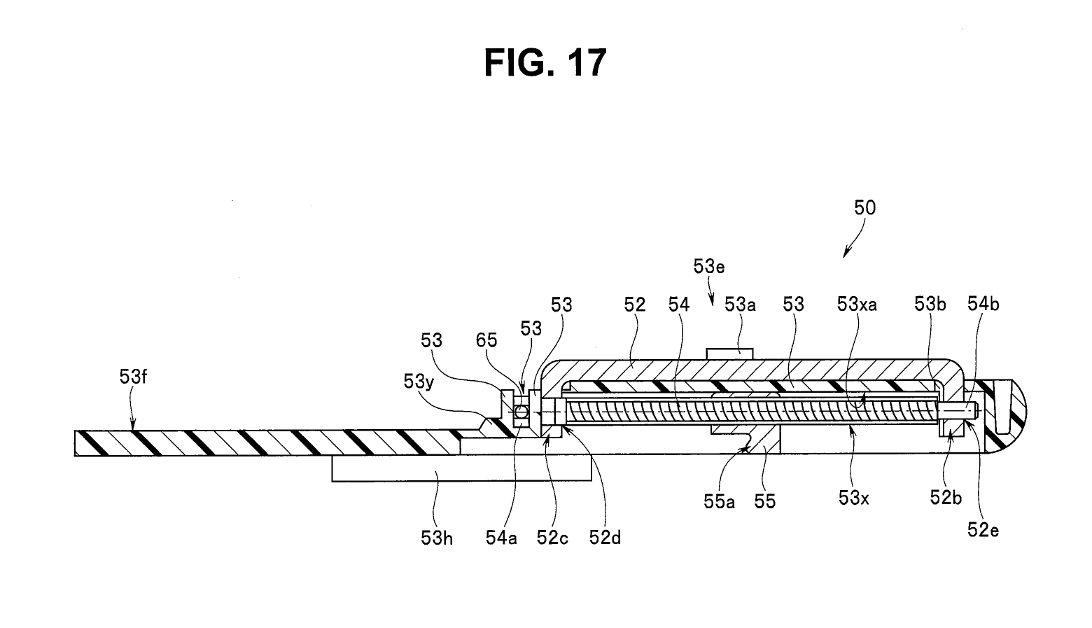

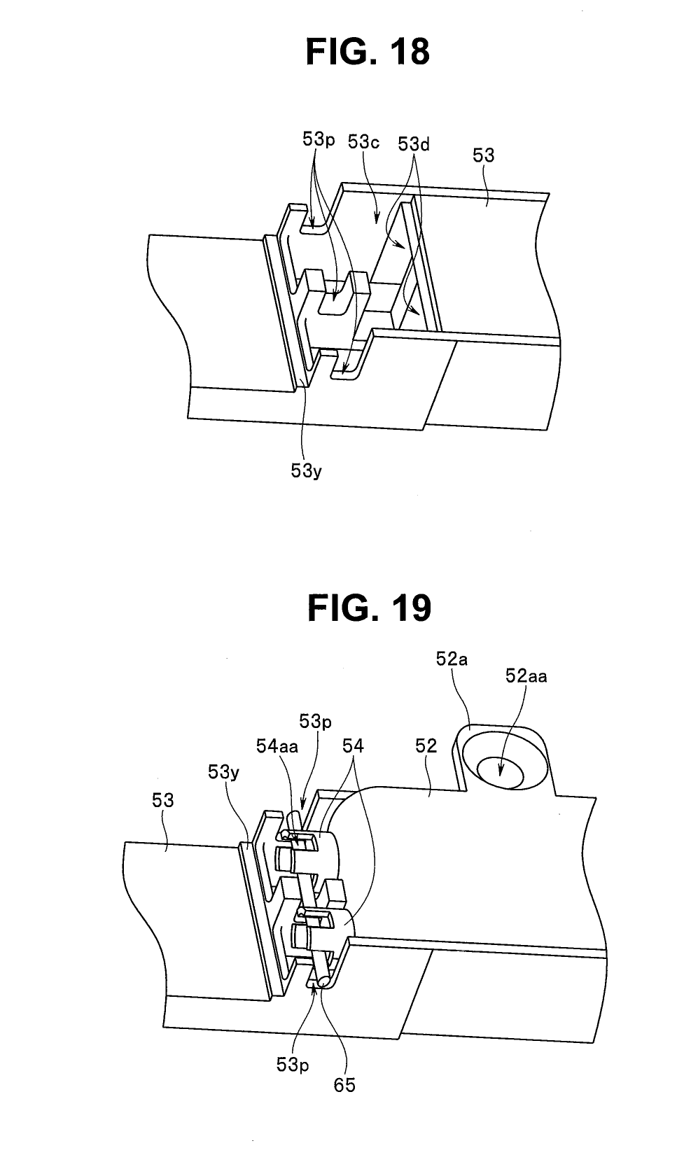

A detailed configuration of the bending-angle adjusting mechanism 50 is explained below with reference to FIG. 9 to FIG. 19. FIG. 9 is a main part enlarged exploded perspective view showing main constituent members of the bending-angle adjusting mechanism in the endoscope in the present embodiment. FIG. 10 is a three-plane drawing conceptually showing a shape of the guide block in the bending-angle adjusting mechanism shown in FIG. 9. FIG. 11 is a four-plane drawing conceptually showing a shape of a bearing plate in the bending-angle adjusting mechanism shown in FIG. 9. FIG. 12 is a diagram conceptually showing disposition of the bending operation mechanism and the bending-angle adjusting mechanism disposed on an inside of the operation section in the endoscope in the present embodiment. FIG. 13 and FIG. 14 are main part enlarged assembled perspective views showing a state in which the bending-angle adjusting mechanism shown in FIG. 9 is assembled. FIG. 13 is a diagram showing a state viewed from an arrow [13] direction shown in FIG. 9. FIG. 14 is a diagram showing a state viewed from an arrow [14] direction shown in FIG. 9. FIG. 15 is a main part enlarged view enlarging and showing a part of the bending-angle adjusting mechanism shown in FIG. 9 and is a diagram mainly showing a coupling structure of the chain and the coupling member. FIG. 16 is a plan view showing one side surface in the bending-angle adjusting mechanism shown in FIG. 9. FIG. 17 is a sectional view taken along line [17]-[17] shown in FIG. 16. FIG. 18 and FIG. 19 are main part enlarged perspective views enlarging and showing a part of FIG. 17. FIG. 18 is a main part enlarged perspective view enlarging and showing a part of the guide block among the constituent members of the bending-angle adjusting mechanism shown in FIG. 9. FIG. 19 is a main part enlarged perspective view showing a state in which two adjustment shafts are built into the bearing plate which is assembled to the guide block shown in FIG. 18 and fixed by a fixing pin.

As shown in FIG. 3 and the like, the bending-angle adjusting mechanism 50 in the present embodiment is provided on the inside of the operation section 3 and is a mechanism unit for setting respective maximum bending angles in the four directions of the upward and downward directions and the left and right directions of the bending section 7. The bending-angle adjusting mechanism 50 is, for example, screwed and fixed to the main frame 31. That is, the bending operation wire 35, which is the towing member coupled to the bending-angle adjusting mechanism 50, is disposed to be movable on the main frame 31.

As shown in FIG. 9 and the like, the detailed configuration of the bending-angle adjusting mechanism 50 is mainly configured by a bearing plate 52, which is a first fixing member and a second fixing member, the guide block 53, two adjusting shafts 54, two stoppers 55, a fixing pin 65, and the like.

The guide block 53 is a guide member including a portion fixed between the adjusting shafts 54 and the bearing plate 52, disposed in parallel to the adjusting shafts 54 and the bearing plate 52, and provided with, in the axial direction (the longitudinal direction) of the insertion section 2, a guide surface (explained in detail below), which is a guide path and a guiding surface for guiding positions of the stoppers 55.

The guide block 53 is formed to include a first guide section 53e and a second guide section 53f. The first guide section 53e is a part that includes a space in which the two adjusting shafts 54 are disposed, guides movement in the axial direction of the two stoppers 55 screwed and disposed in the respective adjusting shafts 54, and restricts rotation around axes of the respective stoppers 55. The second guide section 53f is a part that guides movement in the axial direction of the two coupling members 41 and restricts rotation around axes and side shift of the respective coupling members 41. The adjusting shafts 54 are bar members provided to extend in the axial direction (the longitudinal direction) of the insertion section 2, spiral screw sections (spiral grooves) being formed on circumferential surface in the longitudinal direction of the bar members. The stoppers 55 are adjusting pieces screwed in the screw sections (the spiral grooves) of the adjusting shafts 54, whereby positions in the axial direction on the screw sections (the spiral grooves) of the adjusting pieces are positioned.

More specifically, the first guide section 53e is formed in a substantially box shape, three surfaces of which are formed by wall surfaces to cover outer edges of the spiral grooves of the two adjusting shafts 54 and which has an opening on one surface. Two groove sections 53x are formed in an opening part of the first guide section 53e. On the other hand, the second guide section 53f is formed in a substantially tabular shape and a part of the second guide section 53f is formed in a substantially box shape including a wall section 53h. The groove sections 53x extending in a form in which the two groove sections 53x continue are formed on one surface of the second guide section 53f. The wall surfaces of the three surfaces covering the spiral grooves of the adjusting shafts 54 in the first guide section 53e function as guide surfaces, which are guiding surfaces for guiding movement in the axial direction of the stoppers 55. In this case, a bottom surface of the first guide section 53e is set as a guide surface 53xa (see FIG. 17) in particular. In the second guide section 53f, the two groove sections 53x are traveling surfaces for the chains 32 and the coupling members 41 and are guide surfaces 53xb (see FIG. 16), which are guiding surfaces for the coupling members 41 and the chains 32. That is, in the guide block 53, the guide surfaces 53xa and 53xb, which are a plurality of guiding surfaces, through which the respective chains 32 respectively pass, are integrally provided.

In an intermediate part of the guide block 53, that is, a part overlapping the first guide section 53e and the second guide section 53f, a wall section 53h (see FIG. 13 and the like) is formed to cover parts of both side edge portions of the two groove sections 53x. The wall section 53h is protrudingly provided in a direction orthogonal to the axial direction of the guide block 53 and toward a surface on which the two groove sections 53x are formed.

At both side edge portions of an intermediate part of the first guide section 53e, snap-fit sections 53a projecting in an opposite direction of a projecting direction of the wall section 53h are protrudingly provided. The snap-fit sections 53a are fixing means provided to elastically join and integrate the guide block 53 and the bearing plate 52. Consequently, the snap-fit sections 53a play a role of restricting the stoppers 55 from moving in an orthogonal direction with respect to the guide surface (the two groove sections 53x) of the guide block 53. That is, in the snap-fit sections 53a, the guide block 53 is integrated with the bearing plate 52 screwed and fixed to the main frame 31. Then, the stoppers 55 integrally disposed in the bearing plate 52 via the adjusting shafts 54 are restricted from moving in the orthogonal direction with respect to the guide surface (the two groove sections 53x) of the guide block 53. The bearing plate 52 is a plate member provided in parallel to the axial direction (the longitudinal direction) of the adjusting shafts 54 and both ends of which are formed to be bent in a substantially right-angle direction, the plate member allowing the adjusting shafts 54 to be inserted therethrough at both the ends and rotatably axially supporting the adjusting shafts 54.

Note that the guide block 53 is formed by integral molding using a material having elasticity, for example, a resin material such as polyacetal. In the guide block 53, as explained above, the two groove sections 53x are formed from the proximal end side to the distal end side. The two groove sections 53x are provided to dispose the two chains 32 and the two coupling members 41 concatenated to the respective chains 32 and guide the chains 32 and the coupling members 41 to move conforming to bending operation of the bending operation knobs 22.

That is, in the first guide section 53e, the two adjusting shafts 54 are disposed to be independent from and parallel to each other. Therefore, in the first guide section 53e, the two adjusting shafts 54 are disposed and the two groove sections 53x for guiding the movement in the axial direction of the two stoppers 55 are formed. The two groove sections 53x are concatenated and formed as groove sections for guiding the movement in the axial direction of the two coupling members 41 in the second guide section 53f. That is, the two groove sections 53x are integrally formed to continue from the proximal end side of the first guide section 53e to the distal end side of the second guide section 53f.

In the first guide section 53e, a wall surface 53c for positioning and fixing a bent section 52c on one end side of the bearing plate 52 is formed in an intermediate part and a concave section 53b for fitting and fixing a bent section 52b on the other end side of the bearing plate 52 is formed in a part close to a proximal end (see FIG. 9). On the wall surface 53c, through-holes 53d opened toward the axial direction, through which the adjusting shafts 54 are inserted, are drilled. The through-holes 53d are formed in positions coinciding with through-holes 52d of one end side bent section 52c of the bearing plate 52 in the axial direction.

In a state in which the two adjusting shafts 54 are disposed in predetermined positions on an inside of the guide block 53, the two adjusting shafts 54 are disposed substantially in parallel. In this state, in a part sandwiched between the two adjusting shafts 54, wall sections 53g (see FIG. 16) standing upright in the orthogonal direction from the guide surfaces 53xa and 53xb shown in FIG. 16 and FIG. 17 are extended in the axial direction.

As explained above, the first guide section 53e of the guide block 53 includes spaces (the groove sections 53x) in which the two adjusting shafts 54 are disposed. When the respective adjusting shafts 54 are disposed in the spaces (the groove sections 53x), a spiral groove (a screw section) is formed over a substantially entire length in regions of the respective adjusting shafts 54 located between portions where both ends of the bearing plate 52 bend and face each other (between the one end side bent section 52c and the other end side bent section 52b). Consequently, the spiral grooves of the adjusting shafts 54 are configured to be protected from the outside in substantially all regions in the groove sections 53x of the guide block 53. Therefore, there is no concern that the spiral grooves of the adjusting shafts 54 are, for example, damaged. The movement of the stoppers 55 in the axial direction is guided by three surfaces in the groove sections 53x of the guide block 53. Therefore, it is possible to secure smooth movement without backlash.

In this way, the guide block 53 is formed in an integrated structure for simultaneously guiding traveling of the two chains 32. In this case, the guide surfaces 53xa and 53xb for guiding the movement in the axial direction of the two stoppers 55 and the two coupling members 41 are formed in a shape that makes use of the wall surfaces forming the guide block 53.

Note that a shape of the guide block 53 is generally formed as shown in FIG. 10. In FIG. 10, an alternate long and short dash line indicated by a sign [F] is an axis extending along the wall sections 53g formed between the two groove sections 53x of the guide block 53. The guide block 53 functioning as a component is formed symmetrically with the axis [F] set as a symmetry axis.

On the other hand, as shown in FIG. 8, the chain covers 38 are disposed along both side edges of the guide block 53. In this case, the two chain covers 38A and 38B are disposed to sandwich both side edge portions 53i shown in FIG. 16 on the distal end side of the guide block 53. In an intermediate part of the guide block 53, the two chain covers 38A and 38B are disposed along respective inner walls 53j (see FIG. 16) of wall sections 53h formed at both side edges. On the proximal end side of the guide block 53, the two chain covers 38A and 38B are disposed to sandwich both side edge portions 53k shown in FIG. 16. In this way, the chain covers 38 are provided in vicinities of the plurality of guide surfaces 53xa and 53xb (see FIG. 16 and FIG. 17) in the guide block 53 and function as restricting members that suppress the chains 32 from moving in directions other than the axial direction (the longitudinal direction) of the chains 32.

With such a disposition configuration, the chain covers 38 disposed in a vicinity of a peripheral section of the guide block 53 are positioned and restricted not to shift in position in respective parts and disposed in proper positions such that the chain covers 38 do not bend toward an inner side in a distal end portion near part and a proximal end portion near part of the guide block 53 and do not bend toward an outer side in an intermediate part of the guide block 53.

In the guide block 53, indicators formed from a predetermined form such as characters, an icon, or a pictograph are added to a predetermined position on an outer surface between the concave section 53b and the wall surface 53c. The indicators are indicators for identifying and displaying whether the bearing plate 52 is correctly built into the guide block 53 when the bending-angle adjusting mechanism 50 is in an assembled state.

More specifically, the indicators are a “DR” indicator indicating that the bending-angle adjusting mechanism 50 is a unit corresponding to bending operation in the downward direction and the right direction as shown in FIG. 4 and a “UL” indicator indicating that the bending-angle adjusting mechanism 50 is a unit corresponding to bending operation in the upward direction and the left direction. On the bearing plate 52 side, an opening 52k, which is a display window for displaying the indicators, is provided to correspond to the indicators.

The bending operation mechanism 30 in the endoscope 1 in the present embodiment includes a pair of bending-angle adjusting mechanisms 50 to be respectively adapted to bending operation in the up-down direction and bending operation in the left-right direction for the bending section 7. In this case, the pair of bending-angle adjusting mechanisms 50 is symmetrically disposed with an axis (a sign [H] in FIG. 3) in the major axis direction set as a symmetry axis, for example, on the inside of the operation section 3.

All of the respective constituent members configuring the respective pair of bending-angle adjusting mechanisms 50 are configured to be common to the bending-angle adjusting mechanisms 50. Therefore, the bending-angle adjusting mechanism 50 corresponding to bending operation in the downward direction and the right direction and the bending-angle adjusting mechanism 50 corresponding to bending operation in the upward direction and the left direction are respectively assembled by the same components.

In this case, assembly and disposition of the bearing plates 52 in the respective bending-angle adjusting mechanisms 50 are configured to be different for the bending operation in the downward direction and the right direction (hereinafter referred to as for DR bending) and for the bending operation in the upward direction and the left direction (hereinafter referred to as for UL bending), that is, asymmetrical in the bending operation in the downward direction and the right direction and the bending operation in the upward direction and the left direction.

Accordingly, the indicators are provided in the guide block 53 and the opening 52k, which is the display window, is provided in the bearing plate 52 to make it possible to indicate whether the bearing plate 52 is built into the guide block 53 at the correct position when the bending-angle adjusting mechanisms 50 are in the assembled state.

More specifically, for example, when the bearing plate 52 is assembled to the guide block 53, if the “DR” indicator shown in FIG. 4 is displayed in the opening 52k, it is seen that a unit of the bending-angle adjusting mechanism 50 is the bending-angle adjusting mechanism 50 for the DR bending. Similarly, when the bearing plate 52 is assembled to the guide block 53, if the “UL” indicator shown in FIG. 5 is displayed in the opening 52k, it is possible to easily identify that a unit of the bending-angle adjusting mechanism 50 is the bending-angle adjusting mechanism 50 for the UL bending.

The bearing plate 52 is a supporting member that turnably axially supports respective both end portions of the two adjusting shafts 54. The bearing plate 52 is formed by, for example, bending of a metal plate member or the like or cutting of a metal block or the like. That is, the bearing plate 52 is formed to have a cross section, which is entirely formed in a channel shape (a C shape). Two through-holes 52e that rotatably support the respective other end portions 54b of the two adjusting shafts 54 are drilled in the bent section 52b on the other end side. Two through-holes 52d that rotatably support vicinities of respective one ends (neck sections near head sections 54a) of the two adjusting shafts 54 are drilled in the bent section 52c on one end side. At one side edge portion of a flat section 52f of the bearing plate 52, two protrudingly provided sections 52a, which are projecting sections projecting outward in a direction parallel to the flat section 52f, are formed. In the respective two protrudingly provided sections 52a, through-holes 52aa for inserting through screws 62 in screwing and fixing the bearing plate 52 to a predetermined fixing part of the main frame 31 are drilled. Therefore, the two protrudingly provided sections 52a are parts functioning as fixing sections for fixing the bending-angle adjusting mechanisms 50 to the main frame 31 via the bearing plate 52. Further, in the flat section 52f of the bearing plate 52, the opening 52k is drilled in a part close to one end. As explained above, the opening 52k is the display window for displaying the indicators.

The two through-holes 52aa for screw insertion formed in the bearing plate 52 are formed to be flash with the flat section 52f. This configuration is a measure for setting inserting directions of the two screws 62 in the same direction. By adopting such a configuration, during work for screwing and fixing the bearing plate 52 in the bending-angle adjusting mechanism 50 to the fixing part of the main frame 31, an effect is obtained that it is possible to quickly perform the work without involving re-holding of a tool such as a driver and, therefore, it is possible to contribute to improvement of assemblability.

The bearing plate 52 is formed to have a cross section, which is entirely formed in a channel shape (a C shape) as described above. By adopting such a shape, it is possible to support both ends of the adjusting shafts 54 with one member and it is possible to contribute to improvement of positioning accuracy such as parallelism to the axial direction of the adjusting shafts 54.

Note that a shape of the bearing plate 52 is generally formed as shown in FIG. 11. In FIG. 11, an alternate long and short dash line indicated by a sign [F1] is a center axis extending along the major axis direction of the flat section 52f of the bearing plate 52. The center axis [F1] is an axis coinciding with the axis [F] of the guide block 53 when the bearing plate 52 is attached to the guide block 53. In the bearing plate 52, a shape of the flat section 52f excluding the protrudingly provided sections 52a is formed symmetrically with the axis [F1] set as a symmetry axis.

In FIG. 11, an alternate long and short dash line indicated by a sign [G] is a center axis orthogonal to the major axis direction of the flat section 52f of the bearing plate 52. The two protrudingly provided sections 52a of the bearing plate 52 are formed in positions asymmetrical with the axis [G] set as a symmetry axis. In an example shown in FIG. 11, one protrudingly provided section 52a is formed in a position away from the axis [G] by a distance L3 and the other protrudingly provided section 52a is formed in a position apart from the axis [G] by a distance L4. The distances L3 and L4 are set to be L3#L4. With this configuration, it is possible to secure an interval between the two protrudingly provided sections 52a wide. Therefore, when the bending-angle adjusting mechanisms 50 are fixed to the main frame 31 via the bearing plate 52, it is possible to more surely perform stable fixing.

The bent sections formed at both the ends of the bearing plate 52, that is, the bent section 52c on one end side and the bent section 52b on the other end side are formed in completely the same shape as shown in FIG. 11.

The adjusting shafts 54 are shaft-like members for adjusting respective maximum bending angles in the four directions of the upward and downward directions and the left and right directions by adjusting the positions of the stoppers 55. Therefore, in the adjusting shafts 54, screw groove sections formed by spiral grooves are formed over substantially entire circumferences of shaft section outer circumferential surfaces. Cutout grooves for adjustment 54aa (see FIG. 9 and the like) for rotating shaft sections are formed in the distal end head sections 54a. The cutout grooves for adjustment 54aa are formed to include cutout grooves at an angle interval of approximately 90 degrees in a circumferential direction.

The adjusting shafts 54 are attached in a state in which neck portions of the head sections 54a are rotatably axially supported in the through-holes 52d of the one end side bent section 52c of the bearing plate 52 and the other end portions are rotatably axially supported in the through-holes 52e of the other end side bent section 52b of the bearing plate 52.

The stoppers 55 include female screw sections that screw in the spiral grooves of the adjusting shafts 54. The stoppers 55 are respectively built into respective insides of the two groove sections 53x formed in the first guide section 53e of the guide block 53 in a state in which the stoppers 55 are screwed in the spiral grooves of the adjusting shafts 54. In this state, movement in the axial direction of the stoppers 55 is guided by a wall surface including the guide surface 53xa. When the adjusting shafts 54 are rotated, the stoppers 55 advance and retract in the axial direction of the adjusting shafts 54. In the stoppers 55, the concave sections 55a with which the convex section 41f of the coupling member 41 comes into contact and engages are formed.

The fixing pin 65 is provided as a member for positioning the stoppers 55 in predetermined positions on the adjusting shafts 54. The fixing pin 65 is a rotation preventing member that restricts the adjusting shafts 54 from rotating with the axial direction (the longitudinal direction) as an axis with respect to the guide block 53. The fixing pin 65 is formed by, for example, a bar-like member made of metal and formed in a columnar shape. The fixing pin 65 is configured to restrict the rotation of the adjusting shafts 54 by being inserted through and disposed in the cutout grooves for adjustment 54aa formed in the head sections 54a of the adjusting shafts 54.

Therefore, in the guide block 53, a fixing-pin attaching section 53p for fixing and disposing the fixing pin 65 in a predetermined position is formed in the guide block 53. As shown in FIG. 18, FIG. 19, and the like, the fixing-pin attaching section 53p is formed in a part where the head sections 54a of the adjusting shafts 54 are disposed when the adjusting shafts 54 are attached to the guide block 53 (in FIG. 18, the adjusting shafts 54 are not shown). The fixing-pin attaching section 53p is formed to include a cutout section that can dispose the fixing pin 65 in a direction not parallel to the axial direction of the guide block 53, for example, the orthogonal direction. In other words, as shown in FIG. 19, the cutout section of the fixing-pin attaching section 53p is formed by a wall surface for pressing bases of the head sections 54a and a wall surface formed to be opposed to the wall surface.

With this configuration, in the guide block 53, the head sections 54a of the adjusting shafts 54 are configured to be housed and disposed in the fixing-pin attaching section 53p. In that state, when the fixing-pin attaching section 53p is viewed from a side surface side, in some case, all of (a gap of) the cutout section of the fixing-pin attaching section 53p and (gaps of) the cutout grooves for adjustment 54aa of the head sections 54a of the two adjusting shafts 54 overlap and a through-space is formed. By inserting and disposing the fixing pin 65 in the through-space, it is possible to simultaneously perform rotation restriction around the axes of the two adjusting shafts 54 and it is possible to perform fixing of the two adjusting shafts 54. At this point, the cutout grooves for adjustment 54aa can be fixed to the fixing pin 65 at every rotation angle of 90 degrees (¼ rotation). In this case, in the position adjustment of the stoppers 55 by the adjusting shafts 54, the rotation angle of approximately 90 degrees of the adjusting shafts 54 does not greatly affect the adjustment of the bending angle.

Note that, as the fixing pin 65, a spring pin may be applied and configured to be pressed in and inserted or a parallel pin may be configured to be inserted. The fixing pin 65 needs to be maintained in an inserted state. When the spring pin is applied as the fixing pin 65, the spring pin is inserted while being pressed in. Therefore, the pin after the insertion does not come off. On the other hand, even if the parallel pin is applied as the fixing pin 65, the parallel pin does not come off because of a reason explained below.

That is, after the position adjustment of the stoppers 55 by the adjusting shafts 54, the parallel pin functioning as the fixing pin 65 is inserted into a portion where the fixing-pin attaching section 53p of the guide block 53 and the cutout grooves for adjustments 54aa of the head sections 54a of the two adjusting shafts 54 overlap. The bending-angle adjusting mechanism 50 is assembled. The bending-angle adjusting mechanism 50 assembled in this way is disposed in a predetermined position on the inside of the operation section 3 of the endoscope 1. In this case, exterior member inner surfaces of the main frame 31 and the operation section 3 are disposed in an inserting and removing direction of the fixing pin 65. Consequently, when the bending-angle adjusting mechanism 50 is built into the inside of the operation section 3, the fixing pin 65 cannot be inserted and removed. Consequently, the rotation of the adjusting shafts 54 is always restricted. Therefore, the adjusted positions of the stoppers 55 are maintained.

As shown in FIG. 18, FIG. 19, and the like, in the guide block 53, in order to suppress the adjusting shafts 54 from coming off, a convex section 53y formed to be orthogonal to the axial direction and projecting outward is formed in a part to be an insertion port for the adjusting shafts 54, that is, a part opposed to the through-holes 52d of the bearing plate 52 and on one plane of the second guide section 53f. As shown in FIG. 17 to FIG. 19, a cross section of the convex section 53y is formed to include, on the distal end side of the guide block 53, a slope directed to the plane of the second guide section 53f. On the other hand, on the proximal end side of the guide block 53, that is, a surface opposed to the head sections 54a of the adjusting shaft 54, the cross section is formed by a wall surface orthogonal to the axial direction of the guide block 53. A projection amount of the convex section 53y is set such that the convex section 53y projects higher than a lower edge position of outer circumference edge sections of the head sections 54a of the adjusting shafts 54 disposed in regular positions. The regular positions of the adjusting shafts 54 are positions at a time when the adjusting shafts 54 are built into the bending-angle adjusting mechanism 50 in a state in which both the ends of the adjusting shafts 54 are rotatably supported by the through-holes 52d and 52e of the both end bent sections of the bearing plate 52.

As explained above, the guide block 53 is formed of the material having elasticity. Therefore, when the bending-angle adjusting mechanism 50 is assembled, after the adjusting shafts 54 are inserted through the through-holes 53d of the guide block 53 and the through-holes 52d of the bearing plate 52, end portions of the adjusting shafts 54 are rotatably engaged in the through-holes 52e of the bearing plate 52. Consequently, both the ends of the adjusting shafts 54 are rotatably axially supported by the guide block 53 and the bearing plate 52. At this point, at a stage before the adjusting shafts 54 are completely axially supported in the predetermined positions, outer circumferential edge portions of the head sections 54a of the adjusting shafts 54 come into contact with the convex section 53y and movement in the inserting direction in the axial direction of the adjusting shafts 54 is hindered. However, since the guide block 53 itself has elasticity, if the adjusting shafts 54 are directly pushed in the axial direction, the convex section of the guide block 53 is elastically deformed and allows the head sections 54a of the adjusting shafts 54 to pass. Consequently, it is possible to dispose the adjusting shafts 54 in predetermined regular positions (positions shown in FIG. 19).

On the other hand, in a state in which the adjusting shafts 54 are present in the regular positions shown in FIG. 19 and are not fixed by the fixing pin 65, the adjusting shafts 54 can move in the axial direction. However, in the bending-angle adjusting mechanism 50 in the present embodiment, since the convex section 53y is provided in the guide block 53, even if the adjusting shafts 54 move to positions where the outer circumferential edge portions of the head sections 54a are in contact with the convex section 53y, the adjusting shafts 54 do not further move in a coming-off direction in the axial direction. Therefore, with this configuration, the convex section 53y is a part that plays a role of suppressing coming-off of the adjusting shafts 54 and reducing damage and the like of the spiral groove due to the coming-off.

The bending-angle adjusting mechanism 50 in the present embodiment configured as explained above is generally assembled as explained below. First, the bearing plate 52 is attached to the guide block 53. For the attachment, the one end side bent section 52c of the bearing plate 52 is brought into contact with the wall surface 53c of the guide block 53. The other end side bent section 52b of the bearing plate 52 is fit in the concave section 53b of the guide block 53.

In this state, the respective other end portions 54b of the adjusting shafts 54 are inserted through the through-holes 53d of the wall surface 53c of the guide block 53 via the through-holes 52d of the one end side bent section 52c of the bearing plate 52. Subsequently, the adjusting shafts 54 are pushed in such that the spiral sections of the adjusting shafts 54 fit in the groove sections 53x of the guide block 53. At this point, the stoppers 55 are screwed in the spiral sections of the adjusting shafts 54. The stoppers 55 are disposed in the groove sections 53x of the guide block 53 between the other end side bent section 52b and the one end side bent section 52c of the bearing plate 52. The other end portions 54b of the adjusting shafts 54 are engaged in the through-holes 52e of the other end side bent section 52b of the bearing plate 52.

When the adjusting shafts 54 are inserted, the head sections 54a of the adjusting shafts 54 come into contact with the convex section 53y of the guide block 53 and temporarily hinder the insertion in the axial direction of the adjusting shafts 54. Since the guide block 53 is formed to have elasticity, if the adjusting shafts 54 are directly pushed in the axial direction, the head sections Ma bend the convex section 53y of the guide block 53 downward along the slope of the convex section 53y while climbing over the slope and retract the convex section 53y from an advancing direction of the adjusting shafts 54. Therefore, the adjusting shafts 54 can be inserted in the axial direction without hindrance.

In this way, the adjusting shafts 54 are rotatably axially supported on the bearing plate 52 in parts near both the end portions of the adjusting shafts 54. Note that one adjusting shaft 54 is disposed in each of the two groove sections 53x of the guide block 53. In this case, the two adjusting shafts 54 are disposed in parallel to the two groove sections 53x of the guide block 53. In this state, the adjusting shafts 54 are disposed in a state in which a part of the guide block 53 is sandwiched between the adjusting shafts 54 and the bearing plate 52 that supports both the ends of the adjusting shafts 54. Consequently, the bending-angle adjusting mechanism 50 is formed in an integrated structure as one unit.

Concerning the constituent unit in which the bearing plate 52, the guide block 53, and the adjusting shafts 54 are integrated in this way, if a tool such as a Phillips head screwdriver is inserted into the cutout grooves for adjustment 54aa to regularly and reversely rotate the adjusting shafts 54, the stoppers 55 advance and retract in the axial direction while being guided by the guide surface 53xa.

In endoscopes applied with the bending-angle adjusting mechanism 50 in the present embodiment, maximum bending angles that should be set are respectively different according to uses and types. Therefore, in the bending-angle adjusting mechanism 50, the maximum bending angles in the respective endoscopes are specified by setting positions of the stoppers 55 on the adjusting shafts 54 in predetermined positions.

After the positions of the stoppers 55 on the adjusting shafts 54 are set as appropriate by means explained above, the fixing pin 65 is inserted through and disposed in the cutout grooves for adjustment 54aa of the head sections 54a of the adjusting shafts 54 and the fixing-pin attaching section 53p. Consequently, since the rotation of the respective adjusting shafts 54 is locked, the stoppers 55 are positioned and fixed in the predetermined positions. Therefore, the set maximum bending angles are specified as predetermined values. The positions of the stoppers 55 do not easily deviate.

The endoscope 1 in the present embodiment includes the pair of bending-angle adjusting mechanisms 50 having the configuration explained above in order to be respectively adapted to the bending operation in the up-down direction and the bending operation in the left-right direction for the bending section 7. More specifically, as explained above, the bending-angle adjusting mechanism 50 for the bending operation in the downward direction and the left direction (for the UL bending) and the bending-angle adjusting mechanism 50 for the bending operation in the upward direction and the right direction (for the DR bending) are formed as a pair to configure one unit.

The pair of bending-angle adjusting mechanisms 50 is respectively disposed as shown in FIG. 12 on the insides of the exterior housing 14 and the grasping section housing 15 configuring the operation section 3 of the endoscope 1. That is, as shown in FIG. 12, an alternate long and short dash line indicated by a sign [J] is a center axis extending along the axial direction of the operation section 3. The center axis [J] is an axis passing the support shaft 34 of the bending operation mechanism 30 in the endoscope 1 in the present embodiment and parallel to the center axis of the insertion section 2. The pair of bending-angle adjusting mechanisms 50 is disposed in positions symmetrical with the center axis [J] set as a symmetry axis. Note that the center axis [J] corresponds to the axis [H] in FIG. 3. As explained above, the lines indicated by the signs [A] and [B] shown in FIG. 12 are the parts on the grasping section housing 15 side of the operation section 3. The part indicated by the sign [L] shown in FIG. 12 is present on the extended line of the line [A] and is the part on the one side surface of the exterior housing 14 of the operation section 3. More specifically, the part indicated by the sign [L] shown in FIG. 12 indicates, on the extended line of the line [A], one side surface part of the exterior housing 14 from one end portion of the exterior housing 14 of the operation section 3 to an imaginary line [N] passing the rotation center axes of the bending operation knobs 22 and the sprocket 33 in the bending operation mechanism 30 and orthogonal to the center axis [J]. The part [L] in this range is referred to as first operation section. The part indicated by the sign [M] shown in FIG. 12 is a part on the other side surface of the exterior housing 14 of the operation section 3 on the line [B] side. More specifically, the part indicated by the sign [M] shown in FIG. 12 indicates, on the extended line of the line [B], the other side surface part of the exterior housing 14 from one end portion of the exterior housing 14 of the operation section 3 to the imaginary line [N] passing the rotation center axes of the bending operation knobs 22 and the sprocket 33 in the bending operation mechanism 30 and orthogonal to the center axis [J]. The part [M] in this range is referred to as second operation section.

In other words, the pair of bending-angle adjusting mechanisms 50 is towing-member adjusting sections disposed on the side of the first grasping section and the first operation section and the side of the second grasping section and the second operation section of the main frame 31 to adjust a moving range of the bending operation wire 35 (the towing member).

In FIG. 12, an arrow [K] indicates a direction in which the insertion section 2 is disposed. In FIG. 12, the sign [A] indicates a part equivalent to the first grasping section explained with reference to FIG. 1 and the sign [L] indicates an axis in the major axis direction extending along the side surface on the side where the first operation section, that is, the part on which the user puts the palm is present. In FIG. 12, the sign [B] indicates a part equivalent to the second grasping section explained with reference to FIG. 1 and the sign [M] indicates an axis in the major axis direction extending along the side surface on the side where the second operation section, that is, the side on which the fingers of the user are put.

In this case, on the inside of the operation section 3, one of the pair of bending-angle adjusting mechanisms 50 is disposed on the axis [A] side in the major axis direction extending along the side surface of the part on which the user puts the palm and the other is disposed on the axis [B] side in the major axis direction extending along the side surface on the side on which the fingers of the user are put. More specifically, the bending-angle adjusting mechanism 50 on the one axis [A] side is configured such that the protrudingly provided section 52a (the first projecting section) of the bearing plate 52 is disposed close to the insertion section 2 side centering on the axis [G]. The bending-angle adjusting mechanism 50 on the other axis [B] side is configured such that the protrudingly provided section 52a (the second projecting section) of the bearing plate 52 is disposed close to the opposite side of the insertion section 2 centering on the axis [G].

With this disposition, a part of the bending-angle adjusting mechanism 50 on the one axis [A] side (the side of the first grasping section and the first operation section) is disposed in the exterior housing 14 and the remaining part is disposed in the grasping section housing 15. In this case, the protrudingly provided section 52a (a fixing section to the main frame 31; a first fixing section) of the bearing plate 52 is located on the outside of the exterior housing 14. Therefore, it is possible to easily perform screwing work in the protrudingly provided section 52a.

On the other hand, a part of the bending-angle adjusting mechanism 50 on the other axis [B] side (the side of the second grasping section and the second operation section) is disposed in the exterior housing 14 and the remaining part is disposed in the grasping section housing 15. In this case, the protrudingly provided section 52a (a fixing section to the main frame 31; a second fixing section) of the bearing plate 52 is located on the outside of the exterior housing 14 and is disposed closer to the exterior housing 14 than the bearing plate 52 on the axis [A] side. Nevertheless, the protrudingly provided section 52a (the fixing section to the main frame 31) of the bearing plate 52 on the axis [B] side is also located on the outside of the exterior housing 14. Therefore, it is possible to easily perform screwing work in the protrudingly provided section 52a.

After the various constituent members are disposed on the inside of the exterior housing 14, the grasping section housing 15 is concatenated to the exterior housing 14 to cover the protrudingly provided section 52a of the bearing plate 52 on the one axis [A] side and the protrudingly provided section 52a of the bearing plate 52 on the other axis [B] side.

As explained above, according to the embodiment, the bending-angle adjusting mechanism 50 in the bending operation mechanism 30 in the constituent unit disposed in the operation section 3 of the endoscope 1 is configured to include the guide block 53 and the bearing plate 52 among the constituent members in common Consequently, it is possible to contribute to a reduction in manufacturing cost.

In this case, the guide block 53 is formed symmetrically with the axis [F] (the axis along the wall sections 53g) in the major axis direction set as the symmetry axis. In the bearing plate 52, the shape of the flat section 52f excluding the protrudingly provided sections 52a is formed symmetrically with the center axis [F1] extending along the major axis direction of the flat section 52f set as the symmetry axis. The two protrudingly provided sections 52a of the bearing plate 52 are formed in the positions asymmetric with the center axis [G] orthogonal to the major axis direction of the flat section 52f of the bearing plate 52 set as the symmetry axis. That is, the bearing plate 52 on the axis [A] side and the bearing plate 52 on the axis [B] side are disposed asymmetrically with respect to the axis [G] and, more specifically, disposed in opposite directions concerning the axis [G] direction.

With such a configuration, by differentiating the disposition of the protrudingly provided sections 52a in attaching the bearing plate 52 to the guide block 53, it is possible to form the respective bending-angle adjusting mechanisms 50 for the DR bending and the UL bending to be asymmetrical.

When the pair of bending-angle adjusting mechanisms 50 formed asymmetrically is disposed in the operation section 3, it is possible to retain a state in which the protrudingly provided sections 52a are always exposed to the exterior housing 14. Therefore, it is possible to configure the bending-angle adjusting mechanisms 50 in a configuration that can contribute to improvement of assembly efficiency without hindering screwing work. Compared with the pair of bending-angle adjusting mechanisms 50 configured symmetrically, it is possible to efficiently dispose the bending operation mechanism 30 in the operation section 3 in which the connecting portion between the exterior housing 14 and the grasping section housing 15 is configured obliquely. Therefore, it is possible to suppress an increase in size in the axial direction of the operation section 3.

As the fixing section for fixing the bearing plate 52 to the main frame 31, the protrudingly provided sections 52a in the two places are formed. Since the protrudingly provided sections 52a in the two places are formed asymmetrically with the center axis [G] set as the symmetry axis, it is possible to easily secure an interval between fixing positions wider. Therefore, when the bending-angle adjusting mechanisms 50 are fixed to the main frame 31 via the bearing plate 52, it is possible to surely perform stable fixing.

Further, since the interval between the two protrudingly provided sections 52a can be secured wider, it is possible to secure, for example, fixed receiving sections corresponding to the snap-fit sections 53a of the guide block 53. Therefore, it is possible to contribute to improvement of assemblability.

It is possible to realize the bending-angle adjusting mechanisms 50 of different forms for the DR bending and for the UL bending while achieving common use of components in the bending-angle adjusting mechanisms 50. In this case, the identification indicators are provided in the guide block 53 and the opening 52k is provided in the bearing plate 52. With this configuration, when the bearing plate 52 is assembled to the guide block 53, according to the identification indicator displayed in the opening 52k, it is possible to easily identify whether the bending-angle adjusting mechanism 50 is for the UL bending or for the DR bending.

Note that the present invention is not limited to the embodiment explained above. Various modifications and applications can be implemented within a range not departing from the spirit of the invention. Further, inventions in various stages are included in the embodiments. Various inventions can be extracted according to appropriate combinations in a disclosed plurality of constituent elements. For example, when the problems to be solved by the invention can be solved and the effects of the invention can be obtained even if several constituent elements are deleted from all the constituent elements described in the one embodiment, a configuration from which the constituent elements are deleted can be extracted as an invention.

### INDUSTRIAL APPLICABILITY

The present invention can be applied not only to an endoscope control apparatus in a medical field but also an endoscope control apparatus in an industrial field.

What is claimed is:

1. An endoscope comprising:
a bendable bending section provided in an insertion section inserted into a subject;
an operation section connected to a proximal end portion of the insertion section and grasped by an operator, a region of the operation section being divided into a first operation section side and a second operation section side across a center axis extending in a longitudinal direction;
a grasping section housing configuring a portion adjacent to the insertion section side in the operation section and grasped by the operator;
an exterior housing configuring a portion located on an opposite side of the insertion section side across the grasping section housing in the operation section, a dimension of the exterior housing along the center axis on the first operation section side being set longer than a dimension of the exterior housing along the center axis on the second operation section side, an internal constituent unit for bending the bending section being disposed on an inside of the exterior housing;
a main frame provided across the center axis in the operation section to fix the internal constituent unit;
a first towing member and a second towing member respectively disposed on the first operation section side and the second operation section side and made movable along the center axis with respect to the main frame in order to bend the bending section;
a first-towing-member adjusting section and a second-towing-member adjusting section respectively disposed on the first operation section side and the second operation section side and configured to adjust moving ranges along the center axis respectively in the first towing member and the second towing member;
a first fixing member fixed to the main frame on the first operation section side to fix the first-towing-member adjusting section to the main frame on the first operation section side; and
a second fixing member fixed to the main frame on the second operation section side to fix the second-towing-member adjusting section to the main frame on the second operation section side, wherein
the first fixing member and the second fixing member are formed by flat plates having a same shape extending along respective moving directions of the first towing member and the second towing member, selected to be fixed to the main frame with one end side directed to the insertion section on the first operation section side or fixed to the main frame with another end side directed to the insertion section on the second operation section side, and formed asymmetrically with respect to the center axis orthogonal to a direction in which the first towing member and the second towing member extend such that fixed sections fixed to the main frame are closer to the one end side than the other end side.

2. The endoscope according to claim 1, wherein the fixed section is screwed to the main frame.

3. The endoscope according to claim 1, wherein the fixed section is formed to project in a direction orthogonal to the direction in which the first towing member and the second towing member extend.

4. The endoscope according to claim 3, wherein
the first fixing member and the second fixing member include flat sections, and
two of the fixed sections are formed to project from the flat sections.
